(19) United States
(12) Patent Application Publication
(10) Pub. No.: US 2026/0337135 A1
(43) Pub. Date: Oct. 29, 2026

(54) METHODS OF MAKING ORTHO-DIYNYL ARENES

(71) Applicant: Mississippi State University, Starkville, MS (US)

(72) Inventors: Dennis W. Smith, JR., Braselton, GA (US); Ernesto I. Borrego, Starkville, MS (US); Niroshani S. Abeynayake, Starkville, MS (US); Gustavo Adolfo Munoz, Starkville, MS (US)

(21) Appl. No.: 19/654,970

(22) Filed: Apr. 22, 2026

Related U.S. Application Data
(60) Provisional application No. 63/792,702, filed on Apr. 22, 2025.

(51) Int. Cl. C07C 17/278; C07C 29/58; C07C 41/24; C07D 213/53; C07D 333/18; C07F 7/08

(52) U.S. Cl. CPC C07C 17/278; C07C 29/58; C07C 41/24; C07D 213/53; C07D 333/18; C07F 7/083

(57) ABSTRACT

Methods for the manufacture of bis-ortho-diynylarene (BODA) and mono-ortho-diynylarene (MODA) monomers are described herein. The BODA and MODA monomers can be used in the preparation of polymer networks.

### CROSS-REFERENCE TO RELATED APPLICATION

This application claims the benefit of and priority to U.S. Provisional Patent Application No. 63/792,702, filed on Apr. 22, 2025, the contents of which is hereby incorporated by reference in its entirety.

### FEDERAL FUNDING STATEMENT

This invention was made with government support under Grant No. HQ0860-24-C-7611 awarded by the Department of Defense (DOD). The government has certain rights in the invention.

### BACKGROUND

Carbon-carbon composite (CCC) and polymer matrix composite (PMC) structures are used in a variety of applications including thin film dielectrics, light emitting diodes, inverse carbon opals, three-dimensional (3D) printing, and as precursors to any other suitable carbon structure. CCCs and PMCs can include a polymer network, for example derived from ortho-diynylarene monomers. Producing such monomers can be expensive and require multiple reaction steps.

It would therefore be advantageous to provide improved methods for the manufacture of ortho-diynyl arene monomers and precursors. It would be particularly advantageous to avoid the use of trifluoromethanesulfonyl chloride.

### SUMMARY

An aspect of the present disclosure is a method of making a bis-ortho-diynylarene (BODA), the method comprising: contacting 3,4-dibromoaniline and 1,2-dibromobenzene in the presence of an alkyl nitrite under conditions effective to provide a reaction mixture comprising 3,3′,4,4′-tetrabromo-1,1′-biphenyl; separating 3,3′,4,4′-tetrabromo-1,1′-biphenyl from the reaction mixture; and contacting the 3,3′,4,4′-tetrabromo-1,1′-biphenyl with an alkyl- or aryl-substituted alkyne in the presence of a palladium catalyst to provide the BODA having the structure

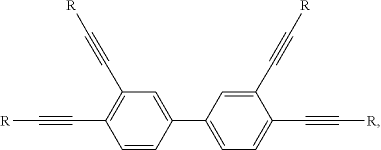

wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.

Another aspect is a method of making a bis-ortho-diynylarene (BODA), the method comprising: brominating a bisphenol of the structure

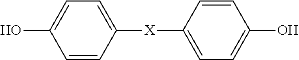

to provide a brominated bisphenol of the structure

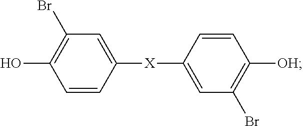

contacting the brominated bisphenol with triflic anhydride to provide a triflated bisphenol of the structure

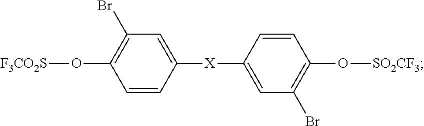

and
contacting the triflated bisphenol with an alkyl- or aryl-substituted alkyne in the presence of a palladium catalyst to provide the BODA having the structure

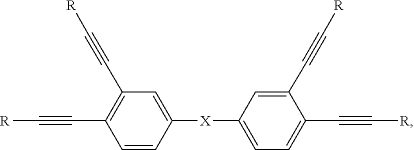

wherein in the foregoing structures, X is a single bond, —O—, or —C(CF₃)—; and R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.

Another aspect is a method of making a mono-ortho-diynylarene (MODA), the method comprising: contacting an aromatic compound of the structure

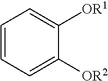

wherein R¹ is hydrogen or methyl; and R² is methyl; with triflic anhydride to provide a triflated aromatic compound having the structure

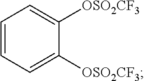

and
contacting the triflated aromatic compound with an alkyl- or aryl-substituted alkyne in the presence of a palladium catalyst to provide the MODA having the structure

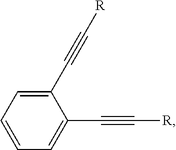

wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.

The above described and other features are exemplified by the following figures and detailed description.

### BRIEF DESCRIPTION OF THE DRAWINGS

The following figures are exemplary embodiments.

FIG. 1 is a chemical scheme illustrating a one-step route to synthesis brominated biaryl compounds.

FIG. 2 is a chemical scheme illustrating triflation of catechol.

FIG. 3 is a chemical scheme illustrating triflation of 3,3′-dibromo-[1,1′-biphenyl]-4,4′-diol.

FIG. 4 is a chemical scheme illustrating triflation of guaiacol and 1,2-dimethoxybenzene.

### DETAILED DESCRIPTION

The present disclosure describes syntheses of ortho-diynyl arene monomers. In an aspect, the synthesis of 3,3′,4,4′-tetrabromo-1,1′-biphenyl can provide bis-ortho-diynylarene (BODA)-biphenyl monomer. In another aspect, the BODA-biphenyl monomer was synthesized through selective o-bromination, followed by quantitative trifluoromethanesulfonate esterification of the corresponding bisphenols, yielding an aryl dibromide ditriflate intermediate. Subsequent Sonogashira-type palladium-catalyzed coupling with phenylacetylene successfully produced the BODA-biphenyl monomer. Advantageously, the presently disclosed methods can reduce the reaction cost and simplify reaction steps in the production of the BODA-biphenyl monomer. A significant improvement is therefore provided by the present disclosure.

Accordingly, an aspect of the present disclosure is a method of making a bis-ortho-diynylarene (BODA). In a specific aspect, the method comprises contacting 3,4-dibromoaniline and 1,2-dibromobenzene in the presence of an alkyl nitrite under conditions effective to provide a reaction mixture comprising 3,3′,4,4′-tetrabromo-1,1′-biphenyl. Exemplary alkyl nitrites can include, but are not limited to, n-pentyl nitrite or isoamyl nitrite. In some aspects, contacting 3,4-dibromoaniline and 1,2-dibromobenzene is further in the presence of a solvent. The solvent, when present, can comprise tetrahydrofuran, dimethylformamide, or acetonitrile. In some aspects, no solvent is present (i.e., the reaction can be conducted in the melt). In some aspects a molar ratio of 1,2-dibromobenzene to 3,4-dibromoaniline can be 1:1 to 10:1. Within this range, the molar ratio can be 2:1 to 8:1, or 3:1 to 6:1, or 4:1 to 5:1.

The reaction of 3,4-dibromoaniline and 1,2-dibromobenzene in the presence of an alkyl nitrite to form 3,3′,4,4′-tetrabromo-1,1′-biphenyl can be conducted at a temperature of, for example, 70 to 100° C. Within this range, the reaction temperature can be, for example, 75 to 95° C., or 80 to 90° C.

In a specific aspect, the reaction of 3,4-dibromoaniline and 1,2-dibromobenzene in the presence of an alkyl nitrite can be in the absence of a solvent, at a temperature of 75 to 95° C., using a molar ratio of 1,2-dibromobenzene to 3,4-dibromoaniline of 4:1 to 5:1, and wherein the alkyl nitrite comprises isoamyl nitrite.

The method further comprises separating the 3,3′,4,4′-tetrabromo-1,1′-biphenyl from the reaction mixture. The separating can comprise, for example, removing residual 1,2-dibromobenzene and, when present, solvent, for example under reduced pressure, and optionally at elevated temperature. In some aspects, the isolated 3,3′,4,4′-tetrabromo-1,1′-biphenyl can be used directly for subsequent method steps without further purification.

The method further comprises contacting the 3,3′,4,4′-tetrabromo-1,1′-biphenyl with an alkyl- or aryl-substituted alkyne in the presence of a palladium catalyst to provide the BODA having the structure

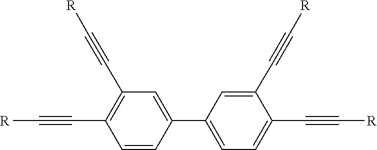

wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof. In some aspects, R can be a C₂₋₁₀ alkyl, for example a C₅ alkyl or a C₈ alkyl. In some aspects, the C₆₋₁₂ aryl can be a phenyl group. In some aspects, the C₃₋₁₂ heteroaryl group can comprise a 5 or 6 membered ring, and can comprise a nitrogen or a sulfur atom. For example, the C₃₋₁₂ heteroaryl group can comprise a thiophene group or a pyridine group. In some aspects, R can be independently at each occurrence

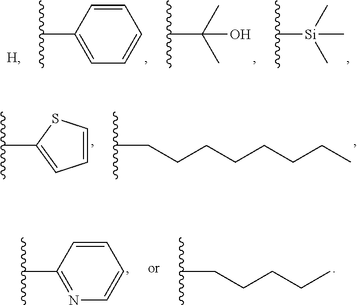

The alkyl- or aryl-substituted alkyne can be of the formula H—C≡C—R, wherein R can be as defined above. Preferably, R can be hydrogen or a phenyl group.

The contacting in the presence of a palladium catalyst can be a Pd-catalyzed Sonagashira coupling reaction. Suitable palladium catalysts for Sonagashira coupling are generally known in the art and can include but are not limited to bis(triphenylphosphine)palladium(II) chloride.

In some aspects, it can be preferable to form the BODA from a bisphenol, for example having the structure

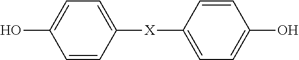

wherein X is a single bond, —O—, or —C(CF₃)—. Other bisphenols not specifically mentioned herein may also be suitable for use in the presently disclosed methods. Prior methods of making BODA monomers from bisphenols typically included 1) bromination via electrophilic aromatic substitution; 2) triflate esterification using trifluoromethanesulfonyl chloride; and 3) Pd cross-coupling to provide the desired BODA monomer. The present inventors have advantageously discovered that ortho-brominated bisphenols can be triflated using triflic anhydride, which is less expensive and easier to handle.

Accordingly, another aspect of the present disclosure us a method of making a bis-ortho-diynylarene comprising selectively brominating a bisphenol to provide a brominated bisphenol of the structure

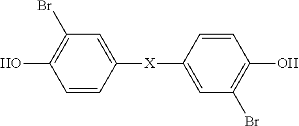

wherein X is as defined above. In some aspects, X is preferably a single bond. Brominating the bisphenol can be by any suitable method for electrophilic addition of a bromine.

The brominated bisphenol can be contacted with triflic anhydride to provide the corresponding triflated bisphenol of the structure

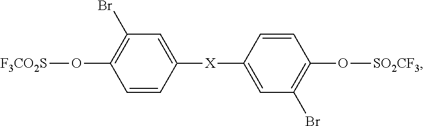

wherein X is as defined above. The triflated bisphenol can be purified and isolated prior to further reaction. For example, the reaction mixture comprising the triflated bisphenol can be washed with aqueous media to remove salts, and organic solvent can be removed under reduced pressed, for example using rotary evaporation. Advantageously, the triflated bisphenol can be provided in high yield, for example greater than 80%, or greater than 85%, or greater than 90%, or greater than or equal to 95%.

The triflated bisphenol can be contacted with an alkyl- or aryl-substituted alkyne in the presence of a palladium catalyst to provide the BODA having the structure

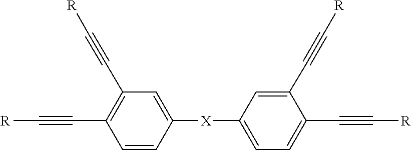

wherein R and X can be as defined above.

Also of interest are di-functional mono-o-diynylarene (MODA) monomers. The present inventors have further advantageously discovered that direct triflation of aromatic compounds containing a methoxy group can be successfully achieved with relatively high yields. Accordingly, yet another aspect of the present disclosure is a method making a mono-ortho-diynylarene (MODA). The method comprises contacting an aromatic compound with triflic anhydride to provide a triflated aromatic compound. The aromatic compound is of the structure

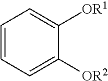

wherein R¹ is hydrogen or methyl; and R² is methyl. In some aspects, R¹ is hydrogen and R² is methyl. In some aspects, R¹ is methyl and R² is methyl.

The corresponding triflated aromatic compound is of the structure

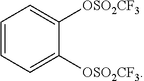

The contacting the aromatic compound with triflic anhydride can be under conditions effective to provide the triflated aromatic compound, for example a temperature of greater than 50° C., or greater than 60° C., or greater than 70° C., or greater than 80° C., for example 70 to 90° C., or 75 to 85° C. Other temperatures may be utilized provided that the reaction proceeds at a sufficient rate. Advantageously, the triflated aromatic compound can be provided in high yields, for example greater than or equal to 80%, or greater than or equal to 85%, or greater than or equal to 90%.

The method further comprises contacting the triflated aromatic compound with an alkyl- or aryl-substituted alkyne in the presence of a palladium catalyst to provide the MODA having the structure

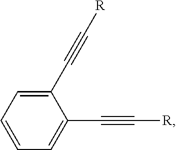

wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof. Preferably, R can be hydrogen or phenyl.

It will be understood that while the method of direct triflation of aromatic compounds containing a methoxy group is described in reference to a mono-ortho-diynylarene, synthesis of bis-ortho-diynylarene will also benefit from this method when a suitable methoxy-containing bisphenol starting material is selected.

The ortho-diynyl arene monomers provided by the methods described herein can be useful in providing a resin. For example, a resin can be formed by thermal cyclopolymerization of the ODA monomers. In some aspects, the thermal cyclopolymerization includes polymerization of one or more BODA monomers. Alternatively, in some aspects, the thermal cyclopolymerization includes copolymerization with one or more BODA monomers and one or more MODA monomers to provide desired rheological properties. In some aspects, the thermal cyclopolymerization proceeds via the radical mediated Bergmann cyclization without catalysts or initiators. Additionally or alternatively, in some aspects, the resin is formed free of condensates or side products. In some aspects, the conditions of the thermal cyclopolymerization are selected to form the resin with any suitable molecular weight. Suitable molecular weights of the resin include, but are not limited to, 1500-25,000 kDa. For example, the thermal cyclopolymerization may include non-oxidative reaction conditions of 1) 250° C. for 4, 6, 12, and 24 hours in diphenyloxide; 2) 210° C. for 4 hours in melt; and 3) 250° C. for 2 hours in melt to form the resin intermediate with a molecular weight of between 1500-25,000 kDa. In some aspects, reaction condition 3) produces a completely cross-linked sample for most ODA copolymer combinations.

In some aspects, the resin can be further processed to form a polymer network. In an aspect, the polymer network can be crosslinker. In some aspects, the polymer network can comprise a structure according to the Formula

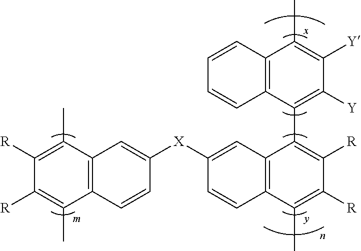

wherein X is O, C(CF₃)₂, or a single bond, R is independently at each occurrence hydrogen, C₂₋₁₀ alkyl, hydroxyl, trimethylsilyl, C₆₋₁₂ aryl, C₃₋₁₂ heteroaryl, or a substituted derivative thereof, Y and Y′ are independently at each occurrence hydrogen or a C₆₋₁₂ aryl group. In an aspect, at least one of Y and Y′ is hydrogen. In an aspect, at least one of Y and Y′ is phenyl. In an aspect, both Y and Y′ are hydrogen. In the polymer network structure, m, n, x, and y represent varying number of repeat units that can be controlled by time and temperature used to form the polymer network.

In some aspects, the polymer network is capable of wetting and holding bundles of carbon fibers free of voids along with corresponding carbon yields. Accordingly, in some aspects, the resin may be combined with carbon fiber bundles prior to processing.

Suitable techniques for forming the polymer network include, but are not limited to, melt processing, thermal curing, or a combination thereof. For example, in some aspects, processing the resin includes thermal processing via extrusion, infusion, coating, micro/nano-molding, or a combination thereof. In some aspects, the processing further includes thermal curing. In some aspects, the thermal curing includes exposing the processed resin to temperatures of at least 450° C. Other suitable techniques include, but are not limited to, solution processing, spin-coating, or micro-molding.

Additionally or alternatively, in some aspects, the resin or polymer network is carbonized. For example, the carbonization can include exposing the processed resin to a temperature of 1000° C. In some aspects, carbonization includes exposing the processed resin to temperatures above 1500° C. In some aspects, exposing the processed resin to a temperature of 1000° C. or more causes pyrolysis, which yields a glassy carbon. In some aspects, the resins or polymer networks according to one or more of the aspects disclosed herein are capable of withstanding high carbonization temperatures while maintaining thermal stability and high performance.

The polymer network formed according to one or more of the aspects disclosed herein includes, but is not limited to, a polymer matrix composite (PMC) and/or a carbon-carbon composite (CCC). These polymer networks are useful for a variety of applications such as, but not limited to, thin film dielectrics, light emitting diodes, inverse carbon opals, three-dimensional (3D) printing, and as precursors to any other suitable carbon structure. In some aspects, the polymer networks form PMCs and/or CCCs with extreme environment stability (e.g., resistance to high heat (e.g., at least 1000° C.), oxidative resistance, chemical resistance). In some aspects, the polymer networks may be used in hypersonic aircraft; missiles; other high speed atmosphere, LEO, or space vehicles; ground based automotive and other high performance carbon applications (high ablation, impact, and wear resistance, etc.); or any other suitable extreme environment application for PMCs and/or CCCs. Additionally or alternatively, in some aspects, the polymer networks may be used in less extreme or non-extreme conditions, such as, but not limited to, electrodes, filters, electronics, or any other suitable carbon application.

Without wishing to be bound by theory, it is believed that the articles and methods disclosed herein provide an unprecedented high yield of CCC structures from carbon fiber and ODA derived polymers. For example, in some aspects, the methods disclosed herein provide a high yield (e.g., over 80%) of carbonized structures after micro- and nano-molding. Once again, without wishing to be bound by theory, it is believed that this high yield represents an extreme and unique processability and performance balance never before applied to polymer matrix composites (PMC) or carbon/carbon composites (CCC). Additionally or alternatively, in some aspects, the monomers disclosed herein exhibit higher carbon content than the existing phenolic resins used as carbon precursor polymer matrices in CCCs, thereby releasing less condensates upon carbonization, and thus requiring less resin infusions and creating a more cost-efficient process for the large scale production of CCCs.

The resins and polymer networks described herein can further be as shown and described in copending U.S. Publication Nos. 2023/0098530 and 2024/0352182, the contents of both of which are hereby incorporated by reference in their entirety for all purposes.

The presently-disclosed subject matter is further illustrated by the following specific but non-limiting examples. The following examples may include compilations of data that are representative of data gathered at various times during the course of development and experimentation related to the presently-disclosed subject matter.

### Examples

In the following examples, commercially available 1,2-dibromobenzene, 3,4-dibromoaniline, n-pentyl nitrite, isopentyl nitrite, and other reagents and solvents were used as received unless otherwise noted. Nuclear magnetic resonance (NMR) experiments (¹H, ¹³C,) were performed on a Bruker AVANCE III 500 MHz instrument. Chloroform-d (CDCl₃) was used as a solvent for NMR experiments, and all chemical shifts are reported in parts per million (δ ppm).

### Synthesis of 3,3′,4,4′-tetrabromo-1,1′-biphenyl via Cadogen Coupling

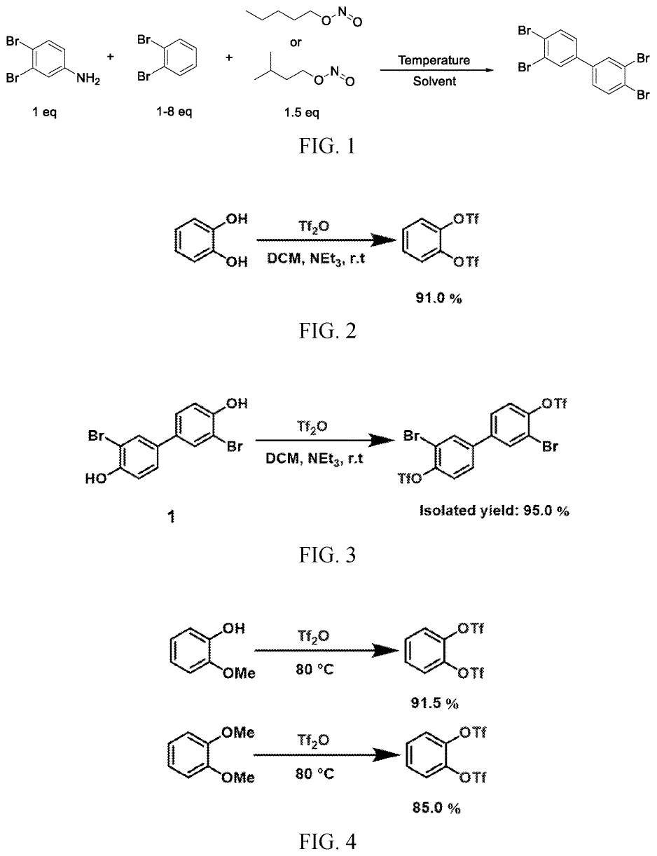

A schematic illustration is shown in FIG. 1. A 100 mL three-neck round-bottom flask was purged with nitrogen and charged with 0.5-5.0 grams of 3,4-dibromoaniline and 0.35-3.5 g of pentyl nitrite or iso amyl nitrite. The flask was charged with 0.47-21.1 g of 1,2-dibromobenzene, and the solution was stirred in an ice bath for 20 minutes. The reaction mixture was then removed from the ice bath and allowed to reach room temperature. The solution was refluxed overnight, and the excess 1,2-dibromobenzene was distilled under reduced pressure. The compound was characterized using NMR spectroscopy. ¹H NMR (CDCl₃, 500 MHz) δ 7.79 (d, J=2.3 Hz, 2H), 7.68 (d, J=8.2 Hz, 2H), 7.32 (dd, J=8.3, 2.2 Hz, 2H). ¹³C NMR (CDCl₃, 190.4 MHz) δ 139.50, 134.29, 132.06, 127.07, 125.70, 124.87.

Reaction conditions were varied to optimize the reaction. Reaction conditions employed are provided in Table 1.

TABLE 1

| | Nitrosating | | Equivalence of | Temperature | Yield of product |
|---|---|---|---|---|---|
| # | agent | Solvent | 1,2-dibromobenzene | °C | mixture (%) |
| 1 | n-pentyl nitrite | — | 2 | 90 | 10 |
| 2 | n-pentyl nitrite | — | 4.5 | 90 | 34 |
| 3 | n-pentyl nitrite | — | 4.5 | 90 | 41 |
| 4 | n-pentyl nitrite | — | 8.0 | 90 | 41 |
| 5 | n-pentyl nitrite | THF (1 mL) | 1.2 | 80 | <5 |
| 6 | Isoamyl nitrite | — | 1 | 90 | trace |
| 7 | Isoamyl nitrite | DMF | 1 | 90 | — |
| | | (1 mL) | | | |
| 8 | Isoamyl nitrite | — | 4.5 | 80 | 48 |
| 9 | Isoamyl nitrite | — | 4.5 | 92 | 49 |
| 10 | Isoamyl nitrite | THF | 4.5 | 80 | 19 |
| | | (2.2 mL) | | | |
| 11 | Isoamyl nitrite | CH₃CN | 4.5 | 80 | 40 |
| | | (2.2 mL) | | | |
| 12 | Isoamyl nitrite | — | 8.0 | 80 | 35 |

### Triflation of Phenol and Methoxybenzene Derivatives

Phenol and its derivatives can react with triflic anhydride (Tf₂O) to obtain the corresponding triflate compound. This reaction occurs in a basic medium at room temperature. As depicted in FIG. 2, catechol can be reacted with Tf₂O to produce the 1,2-bis(triflate)benzene in high (e.g., >90%) yield.

The methodology shown in FIG. 2 offers a viable alternative for triflation reactions, avoiding the use of trifluoromethanesulfonyl chloride, a more expensive and difficult-to-handle reagent. This procedure was demonstrated in the synthesis of 3,3′-dibromo-[1,1′-biphenyl]-4,4′-(bis)triflate (FIG. 3), an intermediate in the preparation of bis-ortho-diynyl arene (BODA). In a two-neck flask, 1.0030 g (2.92 mmol) of compound 1 was dissolved in 20 mL of dry DCM under nitrogen gas. The solution was cooled in an ice bath, and then 1.2 mL of NEt₃ was added, followed by the dropwise addition of 1.1 mL (6.40 mmol) of Tf₂O. The reaction mixture was removed from the ice bath and allowed to warm to room temperature. After 5 hours of reaction, the crude was washed successively with 10% HCl solution, saturated NaHCO₃ solution, and water. The organic layer was dried over Na₂SO₄, and the DCM evaporated. A white crystalline solid was obtained and dried overnight at 60° C. under vacuum (1.6960 g).

The present inventors have also discovered that the direct triflation of aromatic compounds containing a methoxy group can be successfully achieved. FIG. 4 illustrates how, at 80° C., guaiacol and 1,2-dimethoxybenzene can be efficiently converted into 1,2-bis(triflate)benzene with a high yield. This methodology broadens the scope for synthesizing triflate compounds from methoxybenzenes, including certain lignin-derived compounds, thereby expanding the potential sources of triflate intermediates for the synthesis of BODA-type resins. A significant advantage is therefore provided by the present disclosure.

This disclosure further encompasses the following aspects.
Aspect 1: A method of making a bis-ortho-diynylarene (BODA), the method comprising: contacting 3,4-dibromoaniline and 1,2-dibromobenzene in the presence of an alkyl nitrite under conditions effective to provide a reaction mixture comprising 3,3′,4,4′-tetrabromo-1,1′-biphenyl; separating 3,3′,4,4′-tetrabromo-1, l′-biphenyl from the reaction mixture; and contacting the 3,3′,4,4′-tetrabromo-1,1′-biphenyl with an alkyl- or aryl-substituted alkyne in the presence of a palladium catalyst to provide the BODA having the structure

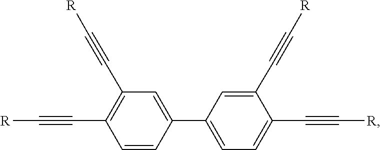

wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.
Aspect 2: The method of aspect 1, wherein the C₅ alkyl nitrite comprises n-pentyl nitrite or isoamyl nitrite.
Aspect 3: The method of aspect 1 or 2, wherein the contacting 3,4-dibromoaniline and 1,2-dibromobenzene is further in the presence of a solvent, preferably a solvent comprising tetrahydrofuran, dimethylformamide, or acetonitrile.
Aspect 4: The method of any of aspects 1 to 3, wherein a molar ratio of 1,2-dibromobenzene to 3,4-dibromoaniline is 1:1 to 10:1, preferably 2:1 to 8:1.
Aspect 5: The method of any of aspects 1 to 4, wherein conditions effective to provide 3,3′,4,4′-tetrabromo-1,1′-biphenyl comprise a temperature of 70 to 100° C., preferably 75 to 95° C., more preferably 80 to 90°.
Aspect 6: A method of making a bis-ortho-diynylarene (BODA), the method comprising: brominating a bisphenol of the structure

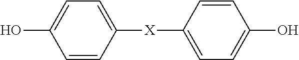

to provide a brominated bisphenol of the structure

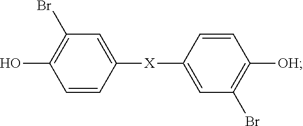

contacting the brominated bisphenol with triflic anhydride to provide a triflated bisphenol of the structure

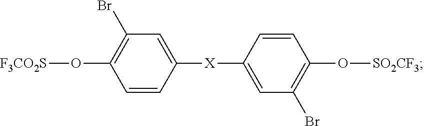

and
contacting the triflated bisphenol with an alkyl- or aryl-substituted alkyne in the presence of a palladium catalyst to provide the BODA having the structure

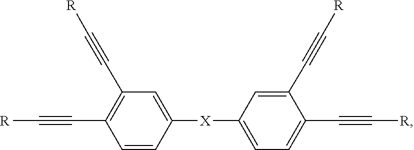

wherein in the foregoing structures, X is a single bond, —O—, or —C(CF₃)—; and R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.
Aspect 7: The method of aspect 6, wherein X is a single bond.
Aspect 8: The method of aspect 6 or 7, wherein R is independently at each occurrence selected from hydrogen,

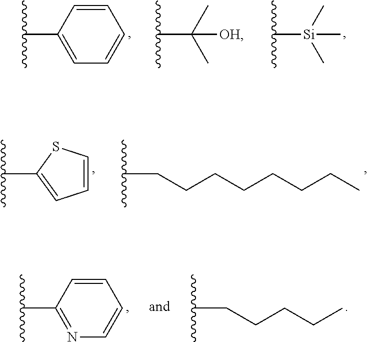

Aspect 9: The method of any of aspects 6 to 8, wherein R is a phenyl group.
Aspect 10: A method of making a mono-ortho-diynylarene (MODA), the method comprising: contacting an aromatic compound of the structure

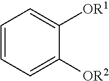

wherein R¹ is hydrogen or methyl; and R² is methyl; with triflic anhydride to provide a triflated aromatic compound having the structure

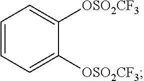

and
contacting the triflated aromatic compound with an alkyl- or aryl-substituted alkyne in the presence of a palladium catalyst to provide the MODA having the structure

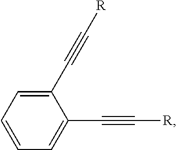

wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.
Aspect 11: The method of aspect 10, wherein R¹ is hydrogen and R² is methyl.
Aspect 12: The method of aspect 10, wherein R¹ is methyl and R² is methyl.
Aspect 13: The method of any of aspects 10 to 12, wherein R is independently at each occurrence selected from hydrogen,

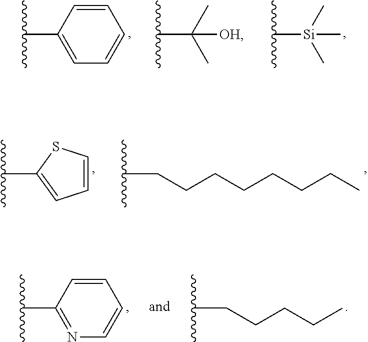

Aspect 14: The method of any of aspects 10 to 13, wherein R is a phenyl group.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through carbon of the carbonyl group.

Unless substituents are otherwise specifically indicated, each of the foregoing groups can be unsubstituted or substituted, provided that the substitution does not significantly adversely affect synthesis, stability, or use of the compound. “Substituted” means that the compound, group, or atom is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g., benzyl), C₇₋₁₂ alkylarylene (e.g., toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O) 2-aryl), or tosyl (CH₃C₆H₄SO₂—), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the compound or group, including those of any substituents.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of making a bis-ortho-diynylarene (BODA), the method comprising:
contacting 3,4-dibromoaniline and 1,2-dibromobenzene in the presence of an alkyl nitrite under conditions effective to provide a reaction mixture comprising 3,3′,4,4′-tetrabromo-1,1′-biphenyl;
separating 3,3′,4,4′-tetrabromo-1,1′-biphenyl from the reaction mixture; and
contacting the 3,3′,4,4′-tetrabromo-1,1′-biphenyl with an alkyl- or aryl-substituted alkyne in the presence of a palladium catalyst to provide the BODA having the structure 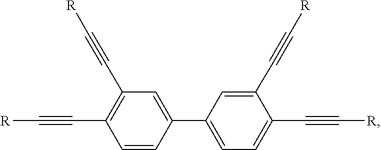
wherein R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.

2. The method of claim 1, wherein the C₅ alkyl nitrite comprises n-pentyl nitrite or isoamyl nitrite.

3. The method of claim 1, wherein the contacting 3,4-dibromoaniline and 1,2-dibromobenzene is further in the presence of a solvent.

4. The method of claim 1, wherein a molar ratio of 1,2-dibromobenzene to 3,4-dibromoaniline is 1:1 to 10:1.

5. The method of claim 1, wherein conditions effective to provide 3,3′,4,4′-tetrabromo-1,1′-biphenyl comprise a temperature of 70 to 100° C.

6. A method of making a bis-ortho-diynylarene (BODA), the method comprising:
brominating a bisphenol of the structure 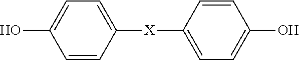
to provide a brominated bisphenol of the structure 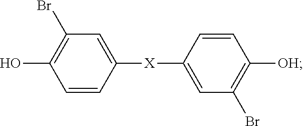
contacting the brominated bisphenol with triflic anhydride to provide a triflated bisphenol of the structure 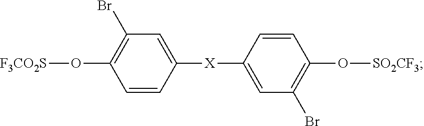
and
contacting the triflated bisphenol with an alkyl- or aryl-substituted alkyne in the presence of a palladium catalyst to provide the BODA having the structure 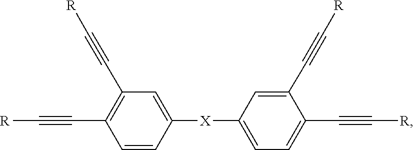
wherein in the foregoing structures,
X is a single bond, —O—, or —C(CF₃)—; and
R is independently at each occurrence hydrogen, C₂₋₁₀ alkyl, hydroxyl, trimethylsilyl, C₆₋₁₂ aryl, C₃₋₁₂ heteroaryl, or a substituted derivative thereof.

7. The method of claim 6, wherein X is a single bond.

8. The method of claim 6, wherein R is independently at each occurrence selected from hydrogen,

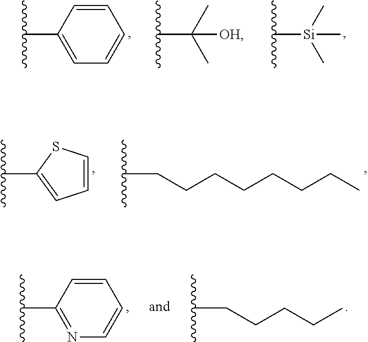

9. The method of claim 6, wherein R is a phenyl group.

10. A method of making a mono-ortho-diynylarene (MODA), the method comprising:
contacting an aromatic compound of the structure 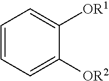
wherein
R¹ is hydrogen or methyl; and
R² is methyl;
with triflic anhydride to provide a triflated aromatic compound having the structure 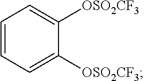
and
contacting the triflated aromatic compound with an alkyl- or aryl-substituted alkyne in the presence of a palladium catalyst to provide the MODA having the structure 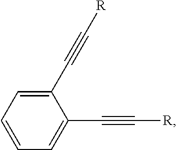
wherein K is independently at each occurrence hydrogen, C₂₋₁₀ alkyl, hydroxyl, trimethylsilyl, C₆₋₁₂ aryl, C₃₋₁₂ heteroaryl, or a substituted derivative thereof.

11. The method of claim 10, wherein R¹ is hydrogen and R² is methyl.

12. The method of claim 10, wherein R¹ is methyl and R² is methyl.

13. The method of claim 10, wherein R is independently at each occurrence selected from hydrogen,

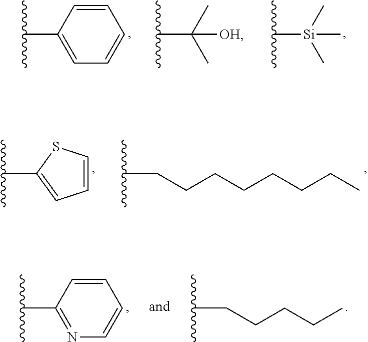

14. The method of claim 10, wherein R is a phenyl group.
